(19) United States
(12) Patent Application Publication
(10) Pub. No.: US 2014/0309541 A1
(43) Pub. Date: Oct. 16, 2014

(54) BLOOD PRESSURE MEASUREMENT DEVICE AND CONTROL METHOD FOR BLOOD PRESSURE MEASUREMENT DEVICE

(71) Applicant: OMRON HEALTHCARE Co., Ltd., Kyoto (JP)

(72) Inventors: Yuki Yamashita, Kyoto (JP); Tatsuya Kobayashi, Kyoto (JP)

(21) Appl. No.: 14/316,062

(22) Filed: Jun. 26, 2014

Related U.S. Application Data
(63) Continuation of application No. PCT/JP2012/077709, filed on Oct. 26, 2012.

(30) Foreign Application Priority Data
Jan. 16, 2012 (JP) 2012-006092

(51) Int. Cl. A61B 5/022

(52) U.S. Cl. CPC A61B 5/02233
USPC 600/485

(57) ABSTRACT

A blood pressure measurement device determines an amplitude and a frequency of a voltage applied to a piezoelectric pump, carries out control so that a voltage at the determined amplitude and frequency is applied to the piezoelectric pump, and calculates a blood pressure value based on a detected cuff pressure during inflation when the cuff pressure is increased by the piezoelectric pump. A control frequency at which a pump efficiency of the piezoelectric pump is maximum in the case where the fluid is supplied to the cuff at a required flow rate during inflation using a predetermined voltage as the voltage is determined, and first control that applies a voltage at the amplitude of the predetermined voltage and at the determined control frequency is carried out. The amount of power consumed can be reduced when increasing the cuff pressure for blood pressure measurement using the piezoelectric pump.

### TECHNICAL FIELD

This invention relates to blood pressure measurement devices and control methods for blood pressure measurement devices, and particularly relates to blood pressure measurement devices suitable for measuring a blood pressure during the inflation of a cuff and to control methods for such blood pressure measurement devices.

### BACKGROUND ART

An electronic blood pressure meter that employs an oscillometric technique is known as a typical electronic blood pressure meter. In an electronic blood pressure meter that employs the oscillometric technique, a manchette containing an air bladder is uniformly wrapped around a part of a body, and changes in the volume of an arterial vessel pressurized by inflating/deflating the air bladder with air are obtained as changes in the amplitude of the pressure in the air bladder (a cuff pressure), which are then used to calculate a blood pressure. To measure the blood pressure accurately while inflating the cuff, it is necessary to properly control the speed at which the pressure within the cuff is increased.

JP 2009-74418A (“Patent Literature 1” hereinafter) proposes a piezoelectric micropump driven using a piezoelectric element, and discusses applying such a pump in an electronic blood pressure meter. Meanwhile, JP 2010-255447A (“Patent Literature 2” hereinafter), JP 2010-162487A (“Patent Literature 3” hereinafter), and so on propose setting a driving frequency according to the material of a piezoelectric element and a diaphragm and carrying out control near the driving frequency.

However, when the pump is driven at a high pressure in this manner, the piezoelectric pump will consume an increased amount of power, and thus a fewer number of blood pressure measurements can be carried out without replacing a battery. Accordingly, it is necessary to improve the inherent mechanical efficiency of the pump.

JP 2006-129920A (“Patent Literature 4” hereinafter) proposes a method for pump flow rate output control using a current, a voltage, a duty, or the like.

### CITATION LIST

### Patent Literature

Patent Literature 1: JP 2009-74418A

Patent Literature 2: JP 2010-255447A

Patent Literature 3: JP 2010-162487A

Patent Literature 4: JP 2006-129920A

### SUMMARY OF INVENTION

### Technical Problem

However, according to the technique of Patent Literature 4, even if the pump flow rate output is the same, there are cases where the energy efficiency of the pump will change depending on a voltage and a frequency, and the maximum energy efficiency of the pump cannot be achieved.

Having been achieved in light of the aforementioned problem, it is an object of the invention to provide a blood pressure measurement device, and a control method for a blood pressure measurement device, capable of reducing the amount of power consumed in the case where a piezoelectric pump is used when increasing a cuff pressure for the purpose of blood pressure measurement.

### Solution to Problem

To achieve the aforementioned object, a blood pressure measurement device according to an aspect of the invention includes a cuff that, when worn on a blood pressure measurement area, pressurizes an artery in the measurement area at the pressure of a fluid in the cuff, a piezoelectric pump that increases the pressure within the cuff, a deflating unit that reduces the pressure within the cuff, a pressure detection unit that detects the cuff pressure that is the pressure within the cuff, and a control unit.

The control unit includes a determination unit that determines an amplitude and a frequency of a voltage applied to the piezoelectric pump, an applied voltage control unit that carries out control so that a voltage at the amplitude and frequency determined by the determination unit is applied to the piezoelectric pump, and a blood pressure measurement unit that calculates a blood pressure value based on the cuff pressure detected by the pressure detection unit during inflation when the cuff pressure is increased by the piezoelectric pump. The determination unit determines a control frequency at which a pump efficiency of the piezoelectric pump is maximum in the case where the fluid is supplied to the cuff at a required flow rate during inflation using a predetermined voltage as the voltage. The applied voltage control unit carries out first control that applies a voltage at the amplitude of the predetermined voltage and at the control frequency determined by the determination unit.

Preferably, the determination unit determines a control voltage at which the pump efficiency is maximum in the case where the fluid is supplied to the cuff at a required flow rate during inflation using a predetermined frequency as the frequency. The applied voltage control unit carries out the first control from the beginning of the inflation to a predetermined time partway through the inflation, and then carries out second control that applies the predetermined frequency and the control voltage determined by the determination unit from the predetermined time to the end of the inflation.

A blood pressure measurement device according to another aspect of the invention includes a cuff that, when worn on a blood pressure measurement area, pressurizes an artery in the measurement area at the pressure of a fluid in the cuff, a piezoelectric pump that increases the pressure within the cuff, a deflating unit that reduces the pressure within the cuff, a pressure detection unit that detects the cuff pressure that is the pressure within the cuff, and a control unit.

The control unit includes a determination unit that determines an amplitude and a frequency of a voltage applied to the piezoelectric pump, an applied voltage control unit that carries out control so that a voltage at the amplitude and frequency determined by the determination unit is applied to the piezoelectric pump, and a blood pressure measurement unit that calculates a blood pressure value based on the cuff pressure detected by the pressure detection unit during inflation when the cuff pressure is increased by the piezoelectric pump. The determination unit determines a control voltage at which a pump efficiency of the piezoelectric pump is maximum in the case where the fluid is supplied to the cuff at a required flow rate during inflation using a predetermined frequency as the frequency. The applied voltage control unit carries out second control that applies the predetermined frequency and the control voltage determined by the determination unit.

Preferably, the determination unit determines a control frequency at which the pump efficiency is maximum in the case where the fluid is supplied to the cuff at a required flow rate during inflation using a predetermined voltage as the voltage. The applied voltage control unit carries out first control that applies a voltage at the amplitude of the predetermined voltage and at the control frequency determined by the determination unit from the beginning of the inflation to a predetermined time partway through the inflation, and then carries out the second control from the predetermined time to the end of the inflation.

Further preferably, the predetermined time is a time at which the cuff pressure reaches a predetermined pressure, the predetermined pressure is determined in advance for each of a plurality of the required flow rates, and the required flow rates are determined in advance based on a size of the cuff, a size of the measurement area, and a state of the cuff as worn on the measurement area.

A control method of a blood pressure measurement device according to yet another aspect of the invention is a control method of a blood pressure measurement device that includes a cuff that, when worn on a blood pressure measurement area, pressurizes an artery in the measurement area at the pressure of a fluid in the cuff, a piezoelectric pump that increases the pressure within the cuff, a deflating unit that reduces the pressure within the cuff, a pressure detection unit that detects the cuff pressure that is the pressure within the cuff, and a control unit.

The control method includes the steps of the control unit determining an amplitude and a frequency of a voltage applied to the piezoelectric pump, carrying out control so that a voltage at the determined amplitude and frequency is applied to the piezoelectric pump, and calculating a blood pressure value based on the cuff pressure detected by the pressure detection unit during inflation when the cuff pressure is increased by the piezoelectric pump. The step of determining includes determining a control frequency at which a pump efficiency of the piezoelectric pump is maximum in the case where the fluid is supplied to the cuff at a required flow rate during inflation using a predetermined voltage as the voltage. The step of carrying out control includes carrying out first control that applies a voltage at the amplitude of the predetermined voltage and at the determined control frequency.

### Advantageous Effects of Invention

According to this invention, the blood pressure measurement device determines an amplitude and a frequency of a voltage applied to the piezoelectric pump, carries out control so that a voltage at the determined amplitude and frequency is applied to the piezoelectric pump, and calculates a blood pressure value based on the cuff pressure detected by the pressure detection unit during inflation when the cuff pressure is increased by the piezoelectric pump. A control frequency at which the pump efficiency of the piezoelectric pump is maximum in the case where the fluid is supplied to the cuff at a required flow rate during inflation using a predetermined voltage as the voltage is determined. First control that applies a voltage at the amplitude of the predetermined voltage and at the determined control frequency is then carried out.

Accordingly, the piezoelectric pump is driven at the control frequency and the predetermined voltage at which the pump efficiency of the piezoelectric pump is maximum using the predetermined voltage as the voltage in the case where the fluid is supplied to the cuff at the required flow rate during inflation, and thus the amount of power consumed can be reduced as compared to a case where the piezoelectric pump is driven at another control frequency and predetermined voltage. As a result, it is possible to provide a blood pressure measurement device, and a control method for such a blood pressure measurement device, that are capable of reducing the amount of power consumed when increasing the cuff pressure for blood pressure measurement using the piezoelectric pump.

### BRIEF DESCRIPTION OF DRAWINGS

FIG. 1 is a perspective view of the outside of a blood pressure meter according to an embodiment of the invention.

FIG. 2 is a block diagram illustrating the overall configuration of the blood pressure meter according to the embodiment.

FIG. 3 is a graph illustrating a pump efficiency when a voltage applied to a piezoelectric pump is varied.

FIG. 4 is a graph illustrating a frequency at which the piezoelectric pump can achieve a maximum flow rate, relative to a voltage value.

FIG. 5 is a graph illustrating a pump efficiency when a voltage of 35 V is applied to the piezoelectric pump.

FIG. 6 is a diagram illustrating variations in the pump efficiency of the piezoelectric pump when controlling a voltage applied during constant speed inflation control.

FIG. 7 is a diagram illustrating variations in the pump efficiency of the piezoelectric pump when controlling a driving frequency of a voltage applied during constant speed inflation control.

FIG. 8 is a diagram illustrating a comparison between pump efficiencies during frequency control and voltage control as well as an applied voltage and a driving frequency.

FIG. 9 is a flowchart illustrating the flow of a blood pressure measurement process executed by a blood pressure meter according to the embodiment.

### DESCRIPTION OF EMBODIMENTS

Hereinafter, an embodiment of the invention will be described in detail with reference to the drawings. Note that identical or corresponding elements in the drawings will be given the same reference numerals, and descriptions thereof will not be repeated.

The following will describe piezoelectric pump driving control when taking an inflation-based measurement using an oscillometric blood pressure meter that takes measurements during inflation as an embodiment of the invention. However, the invention is not limited thereto, and can be applied in another type of a blood pressure meter as long as the blood pressure meter carries out inflation using a piezoelectric pump, such as a blood pressure meter that takes a deflation-based measurement, for example.

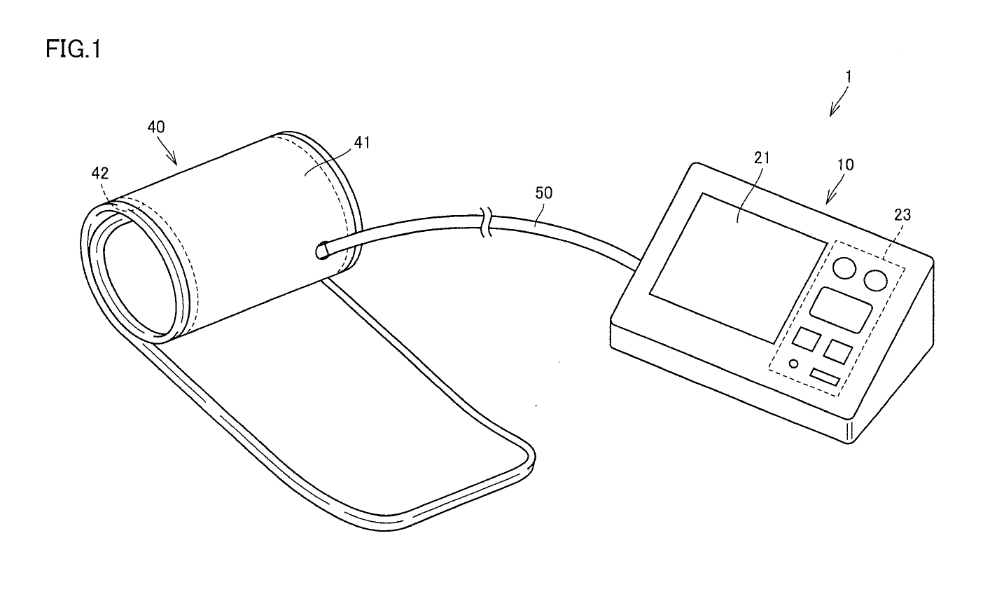

First, the configuration of a blood pressure meter 1 according to this embodiment will be described. FIG. 1 is a perspective view of the outside of the blood pressure meter 1 according to this embodiment of the invention. As shown in FIG. 1, the blood pressure meter 1 according to this embodiment includes a main body 10, a cuff 40, and an air tube 50. The main body 10 includes a box-shaped housing, and a display unit 21 and an operating unit 23 are provided on the top surface thereof. During measurement, the main body 10 is used by being placed on a placement surface such as a table or the like.

The cuff 40 primarily includes a band-shaped and bladder-shaped outer cover 41 and a pressurizing air bladder 42 that is contained in the outer cover 41 and serves as a pressurizing fluid bladder; the cuff 40 has an overall substantially ring-shaped form. During measurement, the cuff 40 is used by being wrapped around and worn on the upper arm of a measurement subject. The air tube 50 connects the main body 10 and the cuff 40, which are configured as separate entities.

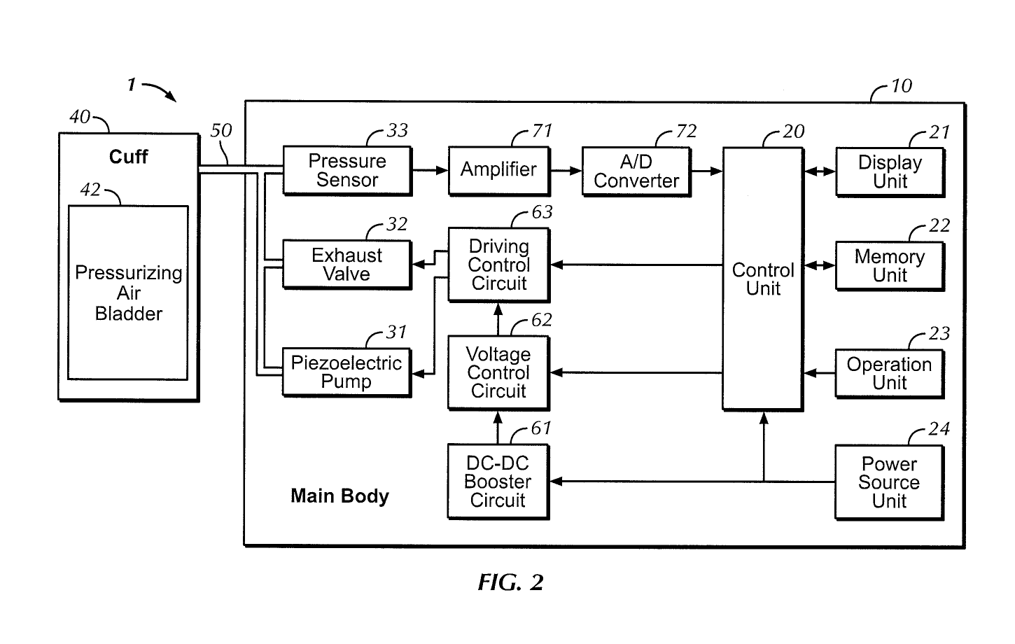

FIG. 2 is a block diagram illustrating the overall configuration of the blood pressure meter 1 according to this embodiment. As shown in FIG. 2, in addition to the display unit 21 and the operating unit 23, the main body 10 includes a control unit 20, a memory unit 22, a power source unit 24, a piezoelectric pump 31, an exhaust valve 32, a pressure sensor 33, a DC-DC booster circuit 61, a voltage control circuit 62, a driving control circuit 63, an amplifier 71, and an AID converter 72. The piezoelectric pump 31 and the exhaust valve 32 correspond to an inflation/deflation mechanism for increasing/decreasing the internal pressure of the pressurizing air bladder 42.

The pressurizing air bladder 42 pressurizes the upper arm when worn thereon, and has an interior space therein. The pressurizing air bladder 42 is connected to the aforementioned piezoelectric pump 31, the exhaust valve 32, and the pressure sensor 33, respectively, via the aforementioned air tube 50. As a result, the pressurizing air bladder 42 is inflated and expands under the driving of the piezoelectric pump 31; the inner pressure is held, the pressurizing air bladder 42 is deflated and contracts, and so on by controlling the driving of the exhaust valve 32.

The control unit 20 is configured of a CPU (central processing unit), for example, and is a unit for controlling the blood pressure meter 1 as a whole.

The display unit 21 is configured of an LCD (liquid-crystal display), for example, and is a unit for displaying measurement results and the like.

The memory unit 22 is configured of a ROM (read-only memory), a RAM (random access memory), or the like, for example, and stores programs for causing the control unit 20 and the like to execute processes for measuring a blood pressure value, stores measurement results, and so on.

The operating unit 23 is a unit for accepting operations made by a measurement subject or the like and inputting such external commands into the control unit 20, the power source unit 24, and the like.

The power source unit 24 is a unit for supplying power to the various units of the blood pressure meter 1, such as the control unit 20 and the piezoelectric pump 31, and is a battery in this embodiment. However, the power source unit 24 is not limited thereto, and may receive power supplied from an external power source such as an AC outlet.

The control unit 20 inputs control signals for driving the piezoelectric pump 31 and the exhaust valve 32 into the voltage control circuit 62 and the driving control circuit 63, respectively, and inputs blood pressure values serving as measurement results into the display unit 21 and the memory unit 22. The control unit 20 also includes a blood pressure information obtainment unit (not shown) that obtains a measurement subject's blood pressure value based on a pressure value detected from the pressure sensor 33 via the amplifier 71 and the A/D converter 72, and the blood pressure value obtained by the blood pressure information obtainment unit is inputted into the aforementioned display unit 21 and memory unit 22 as a measurement result.

Note that the blood pressure meter 1 may also include a separate output unit that outputs a blood pressure value to an external device such as a PC (personal computer), a printer, or the like as the measurement result. For example, a serial communication line, a device that writes to various types of recording media, or the like can be used as the output unit.

The DC-DC booster circuit 61 is a circuit that boosts the voltage of the battery that serves as the power source unit 24 to a voltage suited to the driving of the piezoelectric pump 31.

The voltage control circuit 62 controls the voltage supplied to the piezoelectric pump 31 based on a voltage value indicated by a control signal inputted from the control unit 20.

The driving control circuit 63 controls the piezoelectric pump 31 and the exhaust valve 32 based on a control signal inputted from the control unit 20. Specifically, the driving control circuit 63 controls the frequency of a current supplied to the piezoelectric pump 31 based on a control frequency indicated by the control signal inputted from the control unit 20. In addition, the driving control circuit 63 controls the exhaust valve 32 to open and close based on the control signal inputted from the control unit 20.

The piezoelectric pump 31 is a unit for increasing the internal pressure of the pressurizing air bladder 42 (called the “cuff pressure” as well hereinafter) by supplying air to the interior space of the pressurizing air bladder 42, and the operations thereof are controlled by the aforementioned driving control circuit 63. The piezoelectric pump 31 discharges air at a predetermined flow rate by applying an AC current of a predetermined amplitude V0 at a predetermined driving frequency f0. Note that a sine wave AC may be employed, a square wave AC may be employed, and so on. In the following, the value of a peak-to-peak potential difference Vp-p may be used when discussing the value of a voltage applied to the piezoelectric pump 31. The amplitude is half the value of Vp-p. Relative to Vp-p, the value of the voltage changes in a value range from, for example, −Vp-p/2 to Vp-p/2.

The exhaust valve 32 is a unit for holding the internal pressure in the pressurizing air bladder 42, opening the interior space of the pressurizing air bladder 42 to the exterior and reducing the cuff pressure, and so on, and the operations thereof are controlled by the aforementioned driving control circuit 63.

The pressure sensor 33 detects the internal pressure of the pressurizing air bladder 42 and inputs, into the amplifier 71, an output signal based on the detected pressure. The amplifier 71 amplifies the level of the signal inputted from the pressure sensor 33. The AID converter 72 converts the signal amplified by the amplifier 71 into a digital signal and inputs the generated digital signal into the control unit 20.

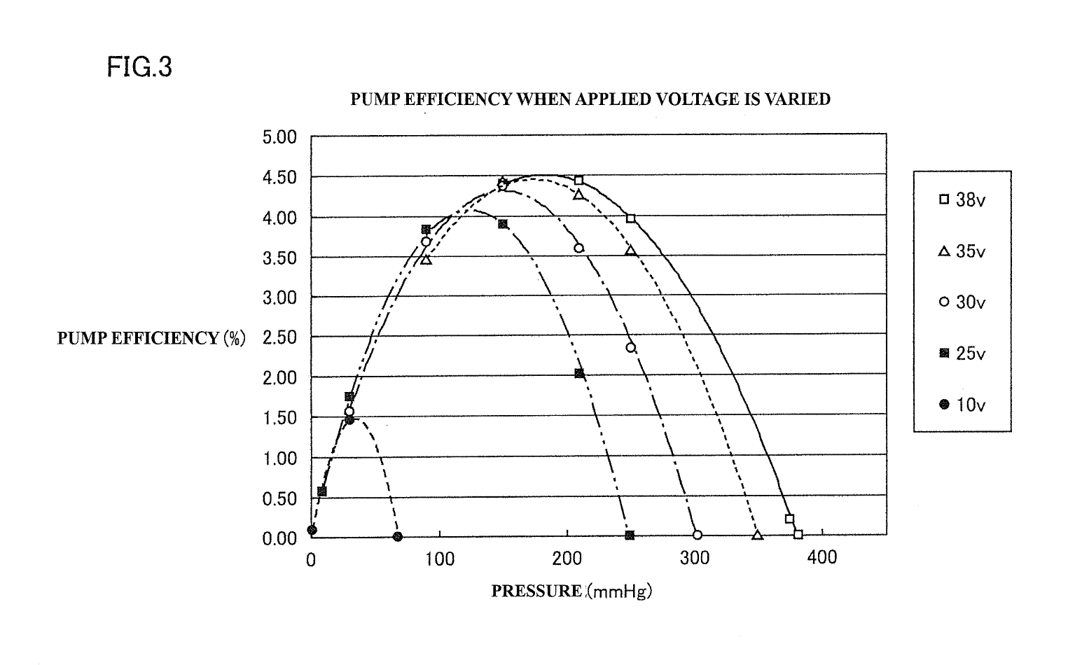

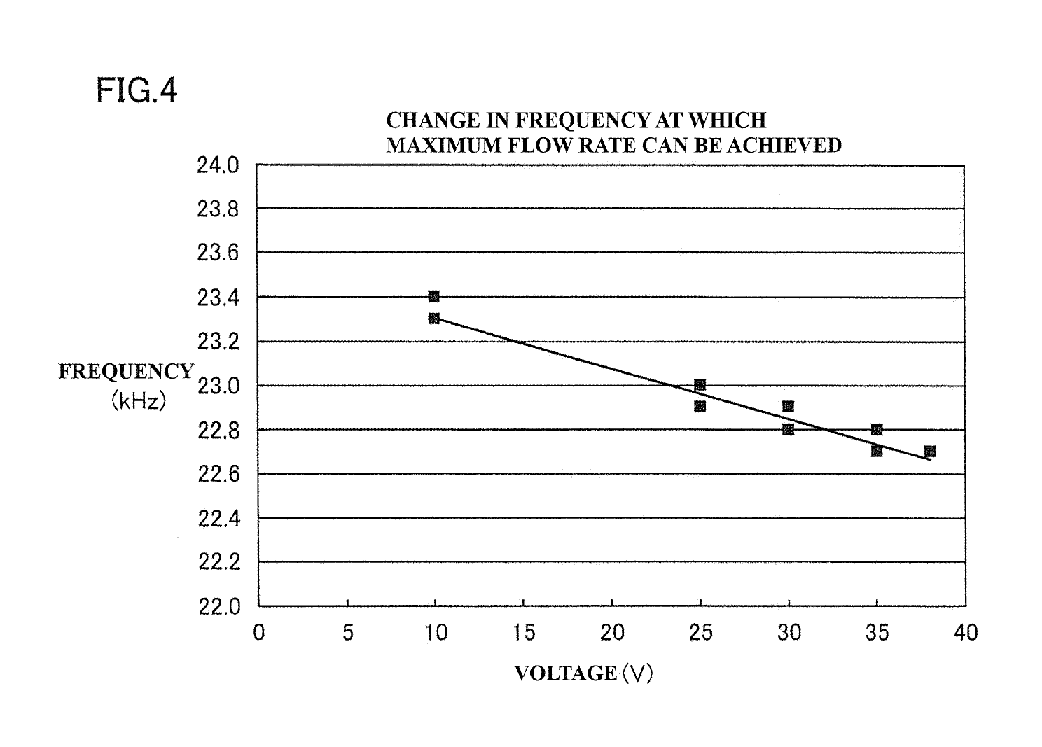

FIG. 3 is a graph illustrating a pump efficiency when a voltage applied to the piezoelectric pump 31 is varied. FIG. 4 is a graph illustrating a frequency at which the piezoelectric pump 31 can achieve a maximum flow rate, relative to a voltage value. The pump efficiency is represented by the ratio of the pump output to the pump input, and is calculated through the formula pump efficiency (%)=pressure (gauge pressure)×flow rate/consumed power.

As shown in FIG. 3, the graphs indicate changes in the pump efficiency accompanying a rise in the cuff pressure when the cuff 40 is inflated, for cases where the voltage applied to the piezoelectric pump 31 is 10 V, 25 V, 30 V, 35 V, and 38 V, respectively.

Meanwhile, FIG. 4 shows that the frequencies at which the maximum flow rate can be achieved when the voltage is 10 V, 25 V, 30 V, 35 V, and 38 V are values of approximately 23.30 kHz, 22.95 kHz, 22.85 kHz, 22.8 kHz, and 22.65 kHz, respectively. Thus the piezoelectric pump 31 is driven at the frequencies shown in FIG. 4 when the corresponding voltage shown in FIG. 3 is applied.

In this manner, the pump efficiency reaches a maximum while the cuff pressure is rising and decreases thereafter, regardless of which voltage is applied. The higher the voltage is, the higher the cuff pressure will be when the pump efficiency is maximum. Furthermore, the higher the voltage is, the higher the pump efficiency will be when the pump efficiency is maximum.

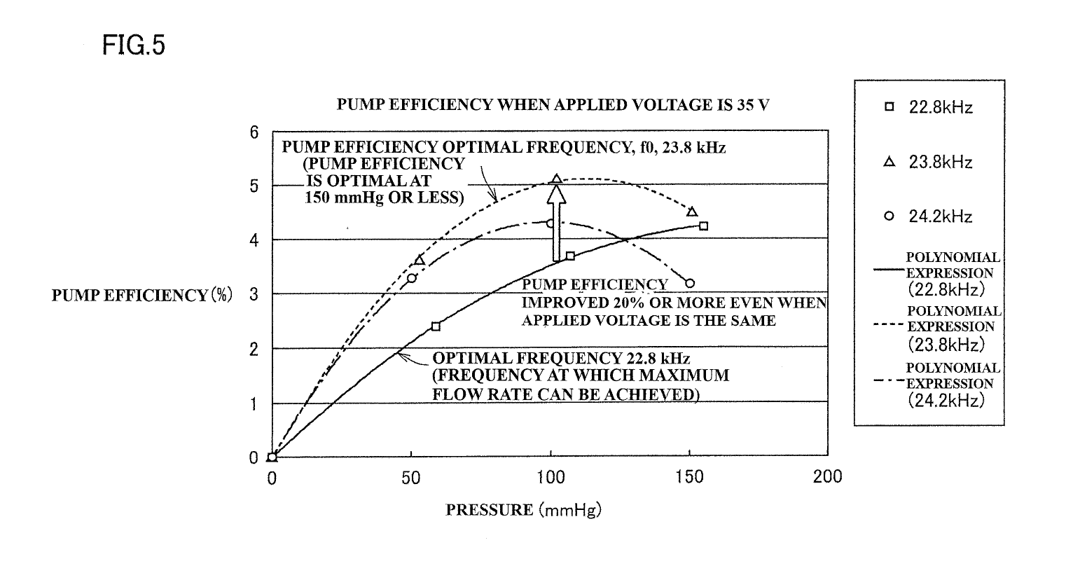

FIG. 5 is a graph illustrating the pump efficiency when a voltage of 35 V is applied to the piezoelectric pump 31. As shown in FIG. 5, when the voltage applied to the piezoelectric pump 31 is set to 35 V, the pump efficiency achieved as the cuff pressure rises when inflating the cuff 40 is improved by 20% or more when an optimal frequency for achieving the maximum flow rate is 23.8 kHz as opposed to 22.8 kHz. This frequency f0 of 23.8 kHz is the frequency that optimizes the pump efficiency until the cuff pressure reaches 150 mmHg.

In this manner, the voltage and driving frequency at which the pump efficiency is optimal differ depending on the range of the cuff pressure. Accordingly, it is conceivable to control the voltage applied to the pump and the driving frequency based on the range of the cuff pressure.

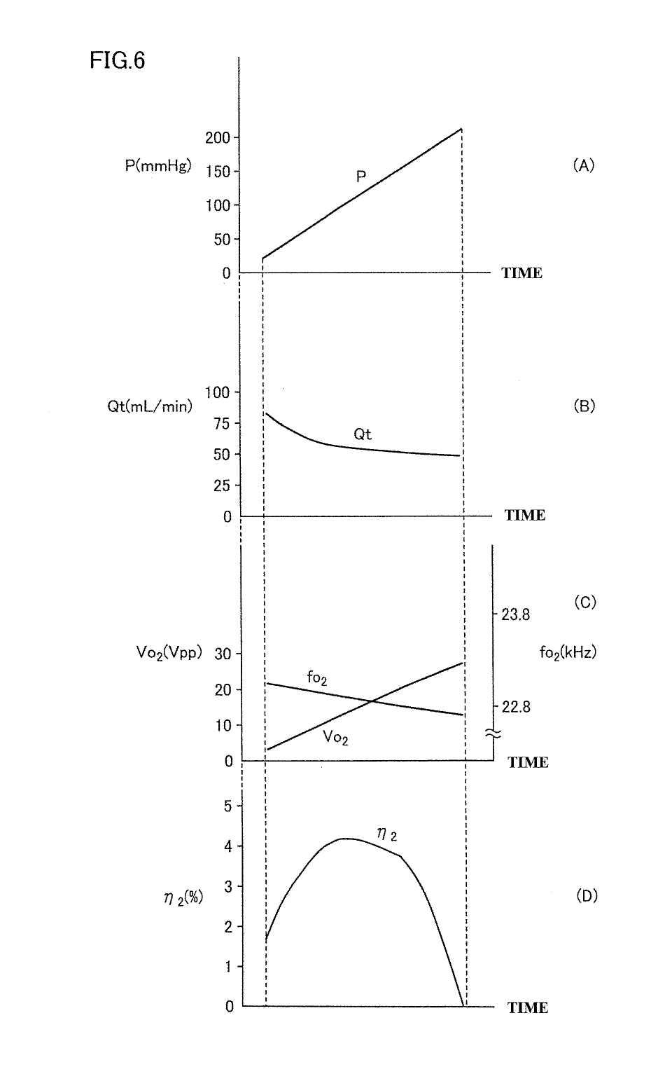

FIG. 6 is a diagram illustrating variations in the pump efficiency of the piezoelectric pump 31 when controlling a voltage applied during constant speed inflation control. As shown in FIG. 6, it is necessary to inflate the cuff at a constant speed in order for the blood pressure meter 1 to measure a blood pressure.

Accordingly, changes in the pump efficiency in the case where a cuff pressure P (mmHg) is increased to 200 mmHg at a constant speed will be described, as indicated in (A) of FIG. 6.

As indicated in (B) of FIG. 6, a flow rate Qt (mL/min) required to increase the cuff pressure P at a constant speed as indicated in (A) of FIG. 6 can be determined when a wrapping state of the cuff and an arm circumference are set. In this manner, the cuff pressure P can be increased at a constant speed by causing the flow rate Qt to decrease slowly.

Next, as indicated in (C) of FIG. 6, in the case where the voltage is controlled in order to cause the piezoelectric pump 31 to discharge air at the flow rate Qt indicated in (B) of FIG. 6, a voltage Vo2 may be increased according to the voltage-flow rate properties of the pump. Note that a driving frequency fo2 is a frequency at which the piezoelectric pump 31 can discharge at the maximum flow rate according to the value of the voltage Vo2, and can be found based on the graph shown in FIG. 4.

As indicated in (D) of FIG. 6, a pump efficiency η2 (%) resulting from driving the piezoelectric pump 31 at the voltage Vo2 and driving frequency fo2 indicated in (C) of FIG. 6 rises during inflation and then drops.

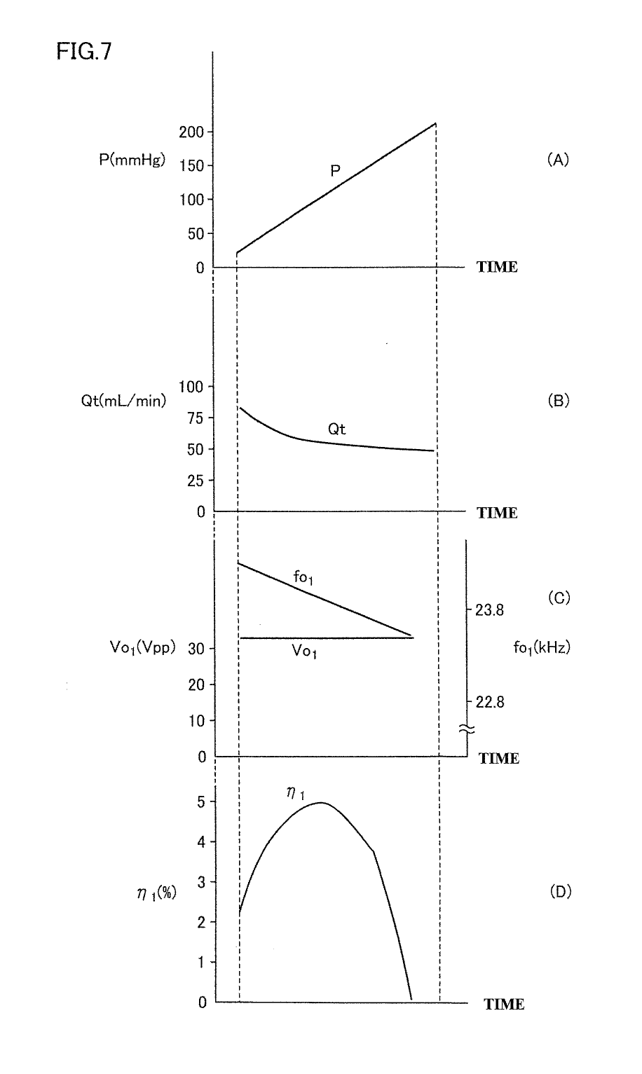

FIG. 7 is a diagram illustrating variations in the pump efficiency of the piezoelectric pump 31 when controlling the driving frequency of the voltage applied during constant speed inflation control. (A) and (B) in FIG. 7 are the same as (A) and (B) in FIG. 6, respectively.

As indicated in (C) of FIG. 7, in order to discharge air from the piezoelectric pump 31 at the flow rate Qt indicated in (B) of FIG. 7, the driving frequency may be controlled so that in the case where a constant voltage Vo1 is applied, a driving frequency fo1 is reduced based on the original voltage-flow rate properties of the pump. Note that although the voltage Vo1 that is applied has a constant value in this embodiment, the invention is not limited thereto, and the voltage may be varied in a set manner.

As indicated in (D) of FIG. 7, a pump efficiency η1 (%) resulting from driving the piezoelectric pump 31 at the voltage Vo1 and driving frequency fo1 indicated in (C) of FIG. 7 rises during inflation and then drops, in the same manner as the case where the applied voltage is controlled as indicated in (D) of FIG. 6.

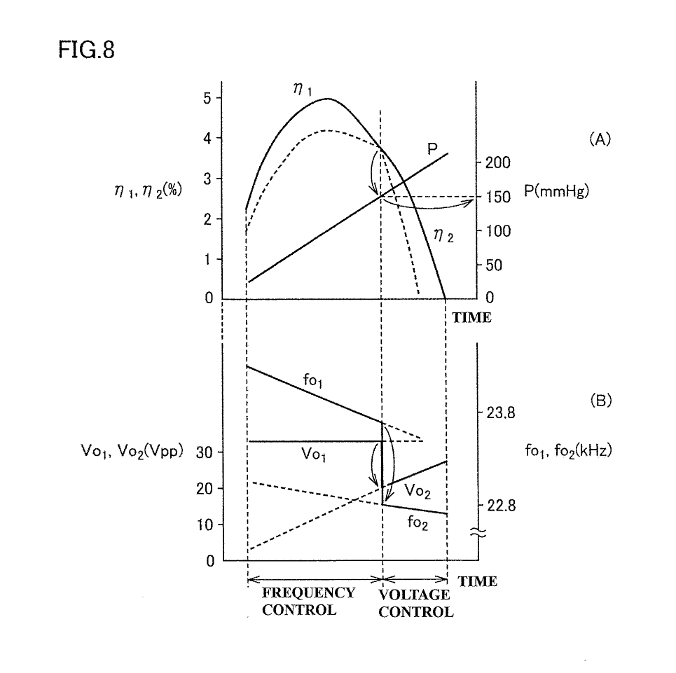

FIG. 8 is a diagram illustrating a comparison between pump efficiencies during frequency control and voltage control as well as an applied voltage and a driving frequency. As indicated in (A) of FIG. 8, the respective pump efficiencies η1 and η2 achieved when carrying out frequency control and voltage control intersect when the cuff pressure P is a pressure P1 (150 mmHg). In other words, frequency control results in a higher pump efficiency value when the cuff pressure is lower than P1. On the other hand, voltage control results in a higher pump efficiency value when the cuff pressure is higher than P1.

Accordingly, as indicated in (B) of FIG. 8, when the cuff pressure is lower than P1, the constant voltage Vo1 is applied and the driving frequency fo1 is controlled, whereas when the cuff pressure is higher than P1, the applied voltage Vo2 is controlled and the driving frequency fo2 at which the maximum flow rate can be achieved is obtained in accordance with the voltage Vo2.

Through this, the piezoelectric pump 31 can be driven through frequency control, which achieves the pump efficiency η1 that is higher than the pump efficiency η2 achieved through voltage control, when the cuff pressure is lower than P1, whereas the piezoelectric pump 31 can be driven through voltage control, which achieves the pump efficiency η2 that is higher than the pump efficiency η1 achieved through frequency control, when the cuff pressure is higher than P1.

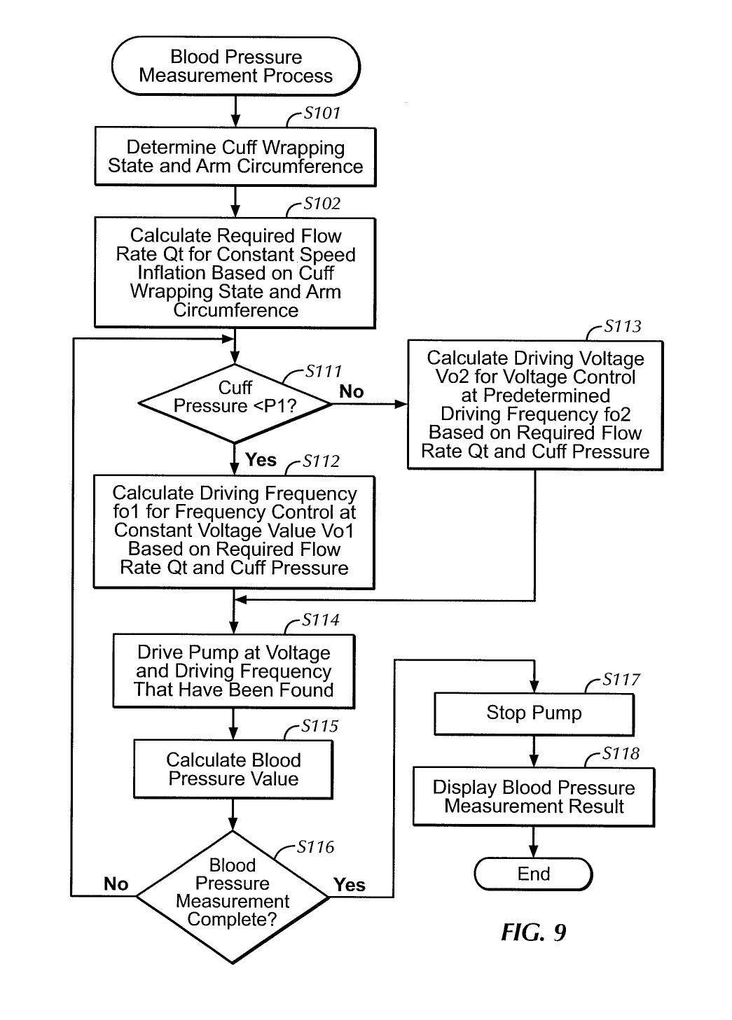

FIG. 9 is a flowchart illustrating the flow of a blood pressure measurement process executed by the blood pressure meter 1 according to this embodiment. As shown in FIG. 9, first, in step S101, the control unit 20 of the blood pressure meter 1 measures the wrapping state of the cuff 40 and the arm circumference. Specifically, an initial inflation is carried out by controlling the piezoelectric pump 31 so that a predetermined amount of air is discharged into the cuff from a state in which there is no pressure in the cuff 40; at this time, the inflation speed is measured, and the wrapping state and arm circumference are estimated based on the measured inflation speed. The method disclosed in International Publication WO 2010/089917 can be given as an example of this method.

Next, in step S102, the control unit 20 calculates the flow rate Qt required to inflate the cuff 40 at a constant speed based on the wrapping state of the cuff 40 and the arm circumference measured in step S101. Specifically, data indicating the graphs shown in (B) of FIGS. 6 and 7 is stored in advance in the memory unit 22 of the blood pressure meter 1 for each of a plurality of sets of wrapping states of the cuff 40 and arm circumferences, and the data indicating the graph of the required flow rate Qt corresponding to the set of the measured wrapping state and the arm circumference is read out from the memory unit 22.

Next, in step S111, the control unit 20 determines whether or not the cuff pressure detected by the pressure sensor 33 and indicated by a signal inputted into the control unit 20 via the amplifier 71 and the A/D converter 72 is less than P1, described with reference to FIG. 8.

In the case where it is determined that the cuff pressure is less than P1 (that is, in the case where a determination of YES is made in step S111), in step S112, the control unit 20 calculates the driving frequency fo1 for frequency control at the constant voltage value Vo1 based on the required flow rate Qt and the current cuff pressure, as described with reference to FIG. 7.

On the other hand, in the case where it is determined that the cuff pressure is not less than P1 (that is, in the case where a determination of NO is made in step S111), in step S113, the control unit 20 calculates the voltage vo1 for voltage control at the predetermined driving frequency fo1 based on the required flow rate Qt and the current cuff pressure, as described with reference to FIG. 6.

Then, in step S114, the control unit 20 sends a signal indicating the voltage value to the voltage control circuit 62 and a signal indicating the driving frequency to the driving control circuit 63 so as to drive the piezoelectric pump 31 at the voltage and driving frequency found in step S112 or step S113.

Next, in step S115, the control unit 20 calculates a blood pressure value according to a conventional method based on changes in the cuff pressure detected by the pressure sensor 33 and indicated by a signal inputted into the control unit 20 via the amplifier 71 and the A/D converter 72.

Then, in step S116, the control unit 20 determines whether or not the blood pressure measurement is complete. In the case where it is determined that the blood pressure measurement is not complete (that is, in the case where a determination of NO is made in step S116), the control unit 20 returns the processing being executed to the process in step S111.

On the other hand, in the case where it is determined that the blood pressure measurement is complete (that is, in the case where a determination of YES is made in step S116), in step S117, the control unit 20 controls the voltage control circuit 62 and the driving control circuit 63 to stop driving the piezoelectric pump 31.

Next, in step S118, the control unit 20 controls the display unit 21 to display the blood pressure measurement result. After step S118, the control unit 20 ends the blood pressure measurement process.

By executing the blood pressure measurement process in this manner, the piezoelectric pump 31 can be controlled so that the cuff 40 can be inflated at a constant speed, and the piezoelectric pump 31 can be controlled so that the pump efficiency improves during the entire course of the constant speed inflation, as described with reference to FIG. 8.

The blood pressure meter 1 according to the embodiments described above achieves effects such as those described below.

(1) The blood pressure meter 1 includes the cuff 40 that, when worn on a blood pressure measurement area, pressurizes an artery in the measurement area at the pressure of the air in the cuff, the piezoelectric pump 31 that increases the pressure within the cuff 40, the exhaust valve 32 that reduces the pressure within the cuff 40, the pressure sensor 33 that detects the cuff pressure that is the pressure within the cuff 40, and the control unit 20.

The control unit 20 determines an amplitude and a frequency of the voltage applied to the piezoelectric pump 31 as indicated in step S112 and step S113 of FIG. 9, carries out control so that a voltage at the determined amplitude and frequency is applied to the piezoelectric pump 31 as indicated in step S114, and calculates a blood pressure value based on the cuff pressure detected by the pressure sensor 33 during inflation when the cuff pressure is increased by the piezoelectric pump 31 as indicated in step S115. The control unit 20 furthermore determines the control frequency fo1 at which the pump efficiency of the piezoelectric pump 31 is maximum in the case where the fluid is supplied to the cuff 40 at a required flow rate Qt during inflation using the predetermined voltage Vo1 as the voltage, and carries out first control that applies a voltage at the amplitude Vo1 of the predetermined voltage and at the determined control frequency fo1, as indicated in step S112 and step S114.

Accordingly, the piezoelectric pump 31 is driven at the control frequency fo1 and the predetermined voltage Vo1 at which the pump efficiency of the piezoelectric pump 31 is maximum using the predetermined voltage Vo1 as the voltage in the case where the fluid is supplied to the cuff 40 at a required flow rate Qt during inflation, and thus the amount of power consumed can be reduced as compared to a case where the piezoelectric pump is driven at another control frequency and predetermined voltage. As a result, the amount of power consumed can be reduced when increasing the cuff pressure for blood pressure measurement using the piezoelectric pump 31.

(2) Meanwhile, the control unit 20 determines the control voltage Vo1 at which the pump efficiency is maximum in the case where the fluid is supplied to the cuff 40 at the required flow rate Qt during inflation using the predetermined frequency fo2 as the frequency, and carries out the first control from the beginning of the inflation to a predetermined time partway through the inflation and then carries out second control that applies the predetermined frequency fo2 and the determined control voltage Vo1 from the predetermined time to the end of the inflation, as indicated in step S113 and step S114 of FIG. 9.

Accordingly, the piezoelectric pump 31 is driven at the control voltage Vo2 and the predetermined frequency fo2 at which the pump efficiency of the piezoelectric pump 31 is maximum using the predetermined frequency fo2 as the frequency in the case where the fluid is supplied to the cuff 40 at a required flow rate Qt during inflation, and thus the amount of power consumed can be reduced as compared to a case where the piezoelectric pump is driven at another control frequency and predetermined voltage. As a result, the amount of power consumed can be reduced when increasing the cuff pressure for blood pressure measurement using the piezoelectric pump 31.

(3) The aforementioned second control may be carried out rather than carrying out the aforementioned first control. Even in such a case, the same effects as those described in (2) above can be achieved.

(4) Furthermore, the predetermined time is a time at which the cuff pressure reaches the predetermined pressure P1 indicated in FIG. 8, the predetermined pressure P1 is determined in advance for each of a plurality of the required flow rates Qt, and the required flow rates Qt are determined in advance based on the size of the cuff 40, the size of the arm circumference serving as the measurement area, and a state of the cuff 40 as worn on the measurement area.

Next, variations on the aforementioned embodiments will be described.

(1) The aforementioned embodiment mentions air as the fluid supplied to the cuff 40 from the piezoelectric pump 31. However, the fluid supplied to the cuff 40 from the piezoelectric pump 31 is not limited thereto, and another fluid, such as a liquid or a gel, may be employed as well. The invention is also not limited to a fluid, and may instead employ uniform particles such as microbeads or the like.

(2) Although the aforementioned embodiment describes the size of the measurement area as corresponding to the arm circumference, the invention is not limited thereto, and different sizes are employed for different measurement areas. For example, in the case where the measurement area is the wrist, the size is the circumference of the wrist.

(3) The aforementioned embodiment describes carrying out frequency control by varying the driving frequency fo1 at the constant voltage value Vo1 in the case where the cuff pressure is less than P1, as described in step S111, step S112, and step S114 of FIG. 9 and with reference to FIG. 8.

However, the invention is not limited thereto, and the frequency control may be carried out by varying the driving frequency fo1 at a voltage value Vo1 that undergoes predetermined changes (increases or decreases, for example) in the case where the cuff pressure is less than P1.

(4) In the aforementioned embodiment, voltage control is carried out by varying the voltage value Vo1 at the driving frequency fo2 that undergoes predetermined changes (decreases, for example) in the case where the cuff pressure is greater than or equal to P1, as described in step S111, step S113, and step S114 of FIG. 9 and with reference to FIG. 8.

However, the invention is not limited thereto, and the voltage control may be carried out by varying the voltage value Vo1 at a constant driving frequency fo1 or at a driving frequency fo1 that undergoes predetermined changes (increases, for example) in the case where the cuff pressure is greater than or equal to P1.

(5) The aforementioned embodiments describe the blood pressure meter 1 as an apparatus invention. However, the invention is not limited thereto, and can also be taken as a control method of the blood pressure meter 1. The invention can also be taken as a control program for the blood pressure meter 1.

Note that the embodiments disclosed above are to be understood as being in all ways exemplary and in no way limiting. The scope of the present invention is defined not by the aforementioned descriptions but by the scope of the appended claims, and all changes that fall within the same essential spirit as the scope of the claims are intended to be included therein as well.

### REFERENCE SIGNS LIST

1 blood pressure meter

10 main body

20 control unit

21 display unit

22 memory unit

23 operation unit

24 power supply unit

31 piezoelectric pump

32 exhaust valve

33 pressure sensor

40 cuff

41 outer cover

42 pressurizing air bladder

50 air tube

61 DC-DC booster circuit

62 voltage control circuit

63 driving control circuit

71 amplifier

72 converter

1. A blood pressure measurement device comprising:
a cuff that, when worn on a blood pressure measurement area, pressurizes an artery in the measurement area at a pressure of a fluid in the cuff;
a piezoelectric pump that increases the pressure within the cuff;
a deflating unit that reduces the pressure within the cuff;
a pressure detection unit that detects a cuff pressure that is the pressure within the cuff; and
a control unit,
wherein the control unit comprises:
a determination unit that determines an amplitude and a frequency of a voltage applied to the piezoelectric pump;
an applied voltage control unit that carries out control so that a voltage at the amplitude and frequency determined by the determination unit is applied to the piezoelectric pump; and
a blood pressure measurement unit that calculates a blood pressure value based on the cuff pressure detected by the pressure detection unit during inflation when the cuff pressure is increased by the piezoelectric pump,
wherein the determination unit determines a control frequency at which a pump efficiency of the piezoelectric pump is maximum in a case where the fluid is supplied to the cuff at a required flow rate during inflation using a predetermined voltage as the voltage; and
wherein the applied voltage control unit carries out first control that applies a voltage at the amplitude of the predetermined voltage and at the control frequency determined by the determination unit from a beginning of the inflation to a predetermined time partway through the inflation.

2. The blood pressure measurement device according to claim 1,
wherein the determination unit determines a control voltage at which the pump efficiency is maximum in a case where the fluid is supplied to the cuff at a required flow rate during inflation using a predetermined frequency as the frequency, and
wherein the applied voltage control unit carries out second control that applies the predetermined frequency and the control voltage determined by the determination unit from the predetermined time to an end of the inflation (step S114).

3. A blood pressure measurement device comprising:
a cuff that, when worn on a blood pressure measurement area, pressurizes an artery in the measurement area at a pressure of a fluid in the cuff;
a piezoelectric pump that increases the pressure within the cuff;
a deflating unit that reduces the pressure within the cuff;
a pressure detection unit that detects a cuff pressure that is the pressure within the cuff; and
a control unit,
wherein the control unit comprises:
a determination unit that determines an amplitude and a frequency of a voltage applied to the piezoelectric pump;
an applied voltage control unit that carries out control so that a voltage at the amplitude and frequency determined by the determination unit is applied to the piezoelectric pump; and
a blood pressure measurement unit that calculates a blood pressure value based on the cuff pressure detected by the pressure detection unit during inflation when the cuff pressure is increased by the piezoelectric pump,
wherein the determination unit determines a control voltage at which a pump efficiency of the piezoelectric pump is maximum in a case where the fluid is supplied to the cuff at a required flow rate during inflation using a predetermined frequency as the frequency, and
wherein the applied voltage control unit carries out second control that applies the predetermined frequency and the control voltage determined by the determination from a predetermined time partway through the inflation to an end of the inflation.

4. The blood pressure measurement device according to claim 3,
wherein the determination unit determines a control frequency at which the pump efficiency is maximum in a case where the fluid is supplied to the cuff at a required flow rate during inflation using a predetermined voltage as the voltage, and
wherein the applied voltage control unit carries out first control that applies a voltage at the amplitude of the predetermined voltage and at the control frequency determined by the determination from a beginning of the inflation to a predetermined time partway through the inflation.

5. The blood pressure measurement device according to claim 2,
wherein the predetermined time is a time at which the cuff pressure reaches a predetermined pressure,
wherein the predetermined pressure is determined in advance for each of a plurality of the required flow rates, and
wherein the required flow rates are determined in advance based on a size of the cuff, a size of the measurement area, and a state of the cuff as worn on the measurement area.

6. A control method for a blood pressure measurement device, the blood pressure measurement device comprising: a cuff that, when worn on a blood pressure measurement area, pressurizes an artery in the measurement area at a pressure of a fluid in the cuff; a piezoelectric pump that increases the pressure within the cuff; a deflating unit that reduces the pressure within the cuff; a pressure detection unit that detects a cuff pressure that is the pressure within the cuff; and a control unit, the control method comprising the steps of:
determining an amplitude and a frequency of a voltage applied to the piezoelectric pump;
carrying out control so that a voltage at the determined amplitude and frequency is applied to the piezoelectric pump; and
calculating a blood pressure value based on the cuff pressure detected by the pressure detection unit during inflation when the cuff pressure is increased by the piezoelectric pump,
wherein the step of determining comprises determining a control frequency at which a pump efficiency of the piezoelectric pump is maximum in a case where the fluid is supplied to the cuff at a required flow rate during inflation using a predetermined voltage as the voltage, and
wherein the step of carrying out control comprises carrying out first control that applies a voltage at the amplitude of the predetermined voltage and at the determined control frequency from a beginning of the inflation to a predetermined time partway through the inflation.

7. The blood pressure measurement device according to claim 4,
wherein the predetermined time is a time at which the cuff pressure reaches a predetermined pressure,
wherein the predetermined pressure is determined in advance for each of a plurality of the required flow rates, and
wherein the required flow rates are determined in advance based on a size of the cuff, a size of the measurement area, and a state of the cuff as worn on the measurement area.
